(19) United States
(12) Patent Application Publication
(10) Pub. No.: US 2009/0053260 A1
(43) Pub. Date: Feb. 26, 2009

(54) ALGAE ESSENCE NUCLEIC ACID FLUID CONCENTRATION PREPARATION METHOD

(75) Inventors: Shih-Hsiung Chan, Taoyuan County (TW); Yen-Chun Chen, Tainan County (TW)

Correspondence Address: WPAT, PC, 7225 BEVERLY ST., ANNANDALE, VA, 22003, US

(73) Assignees: Ying-Yu Hung, Taipei City (TW); GREEN EARS BOUNTIFUL INTERNATIONAL CO. LIMITED, Taipei City (TW)

(21) Appl. No.: 12/195,742

(22) Filed: Aug. 21, 2008

(30) Foreign Application Priority Data
Aug. 22, 2007 (TW) 096131039
Aug. 6, 2008 (TW) 097129787

(51) Int. Cl. A61K 36/05; C07H 21/00

(52) U.S. Cl. 424/195.17; 536/23.6

(57) ABSTRACT

The present invention discloses an alga essence nucleic acid fluid concentration method, to apply the leaching function principle with the operation of freezes defreeze to achieve the goal of extract nucleic acid from the concentrated chlorella. Because of above operation steps with this invention, it will obtain better density of alga nucleic acid fluid. This invention is also to prepare above mentioned particular method of concentrate the alga nucleic acid and produce the alga nucleic acid fluid. The alga nucleic acid fluid may maintain it sweet flavor, and the alga nucleic acid fluid assumes the amber leaning to green color, it is sweet flavor and purely natural taste.

### BACKGROUND OF THE INVENTION

1. Field of the Invention

The present invention is generally related to the nucleic acid fluid from the kind of chlorella (the chlorella hot water to extract “Chlorella Extracts; C.E.; C.G. F.”) Making use of the quality of the solute change in which is produced from leaching action, extraction of the essence in the alga contain the nucleic acid is designated as achievement purpose from operation of freezing and thawing.

2. Description of the Prior Art

Chlorella (Chlorella sp.) It is a kind of unicellular alga plant in fresh water, it was discovered by the Dutch microbiologist “M W Bei Yonick” in 1890, the size is similar to the red corpuscle of the mankind, it can be observed through the microscope, generally being called “green blood corpuscle”, it is the unicellular plant which does not have maneuverability. According to the classification in botany, the chlorella is spherical monad plankton from the fresh water; mainly it is globose or is elliptical shape. Photosynthesis efficiency of the alga is several dozen faster or more than the other plant, also the chlorophyll which contained more abundant than the other plant. Simultaneously, the natural chlorella having the special separation structure which the other photosynthesis living things does not have, the new born natural alga absorbs nutrition and light energy from underwater and to grow, which it matures to blast cell from 20 to 24 hours and furthermore it will separates to four new cells, such a quick speed propagation, is because where the abundant special growth stimulus hormone is included.

The alga includes 55% or more quantity of the vegetable protein, it is something rejoices by the vegetarians. In the alga it included nutrition component such as the chlorophyll and the vegetable fiber, vitamin A, B, C, D and E, the nicotinic acid, folic acid, calcium, iron, magnesium, and various mineral substance and amino acid etc. Furthermore as for the chlorella it will offer the vegetarian whom easily to lack the vitamin B group and, especially vitamin B12; The vitamin B12 it is the important substance which maintains the health of the red corpuscle and nervous system, it is difficult to find in the general vegetables and fruits, but in 5 grams of green algae it contains 4 milligrams of vitamin B12, in which, green algae nucleic acid included the higher quantity than the other food, an attached table as follows.

TABLE 1
The quantity of nucleic acid is included of various foods

| | Food name | | | | | | | | | |
|---|---|---|---|---|---|---|---|---|---|---|
| | Green | Sea | | Bonito | | | Green | | Salmon | Blue |
| | algae | cucumber | Sardine | paragraph | Salmon | Yeast | onion | Egg | extract | alga |
| Content | 13000 | 3605 | 539 | 907 | 289 | 1399 | 78 | 86 | 10600 | 4600 |
| nucleic | DNA&RNA | | | | | RNA | | | DNA | |
| acid | | | | | | | | | | |
| mg/100 g | | | | | | | | | | |

The alga is a kind of alkaline food, long term in take it, can adjust one's physique, many nutrition component which are included in it can helps adjustment of physiological function, maintains digestion performance, promotion of health and beauty care, strength physical ability, health maintenance and longevity prolongation of life, furthermore may take as nourishment for pre-natal, post-natal or after illness, at present time many countries are doing research of the chlorella, to mass produce it and develop into the products to sale.

In the chlorella includes alga essence (Chlorella Extracts C.E.; C.G. F.), is also known as the alga growth stimulation factor, it is the essence of the alga. In every 100 grams of alga usually contains only 4˜5 grams of alga concentrated essence fluid. And in the alga concentrated essence fluid includes the Nucleic Acid, Nucleotide, Folic Acid, Niacin, Lysine, Alanine, Glycine, Praline, Glutamic Acid, Aspartic Acid, Punting Ten, Small Molecular Protein, the water soluble Vitamin and Mineral Substance, is a similar material and ingredient with the animal placenta element. Therefore, it is also named as plant placenta element; and it may stimulate the alga to grow faster.

Generally during the process of concentration and extraction of alga essence, this concentration procedural is a key method to increase the application scope and the commercial value of the alga.

The present technology of concentration procedural is to heat up the alga directly, and evaporate part of moisture to achieve the goal of concentration, and it may be used with the related auxiliary depressurize equipment to increase the efficiency of this part of process.

Due to the long heating process of alga essence fluid, the destruction of the complicated nutrition ingredient is unavoidable. There is unpredictable reaction between various ingredients during the process. Furthermore the long heating process reduces the luster and flavor of the alga concentration fluid. The fact that to solve the fault of the above-mentioned traditional alga essence fluid concentration method and to offer the concentrated method to extracting the nucleic acid include within alga is immediate urgent matter.

### SUMMARY OF THE INVENTION

According to the background of this application, an alga essence nucleic acid fluid concentration method is developed and disclosed.

The main purpose of this invention is to solve the fault from above mentioned traditional alga essence fluid concentration method. It is to offer the concentration method to extract its content of nucleic acid from alga, by applying the leaching function will create the characteristic of solute transformation phenomena, the refrigeration method will achieve it purpose of extract the nucleic acid ingredient from the concentrated alga essence fluid. This invention produces the alga nucleic acid fluid with it color and it luster leaning green with the pure natural flavor, and sweet taste.

In order to achieve above goal, by applying the method with this invention, from chlorella to extract nucleic acid, include following; First, the formation of the chlorella mixture fluid, which made possible by at least intermix with the raw materials of chlorella and to infiltration with the mixed liquid to form the chlorella mixture fluid. Next, process of extraction is proceed, to heats the chlorella mixed liquid evenly at approximately from between 90 degree centigrade up to 120 degrees, and to maintain it for a period of time, then cooling the heated chlorella mixed fluid, it forms the chlorella extracted fluid. Continuously, the dregs and the suspended matter in the chlorella extract are removed and only the chlorophyll is left to form the prototype product of alga essence nucleic acid. Lastly, at least once the leaching process is done, depending on this leaching action to freezes and at the same time nature melting the prototype product of the alga essence nucleic acid fluid, and to complete the concentrated alga essence nucleic acid fluid.

### BRIEF DESCRIPTION OF THE DRAWINGS

FIG. 1 is a flow chart of the alga essence nucleic acid fluid concentrated production method according to the first embodiment of the present invention;

FIG. 2 is a flow chart of the alga essence nucleic acid fluid concentrated production method according to the second embodiment of the present invention;

FIG. 3 is a flow chart of the alga essence nucleic acid fluid concentrated production method according to the third embodiment of the present invention;

FIG. 4 is a operating procedure of the alga essence nucleic acid fluid concentrated production method according to the present invention; and

FIG. 5 is during the concentration stages where collected the leaching solution at the time of each stage the related figure of the solution include it quantity and the time according to the present invention.

### DESCRIPTION OF THE PREFERRED EMBODIMENTS

What is probed into the invention is an alga essence nucleic acid fluid concentration method. Detail descriptions of the structure and elements will be provided as followed in order to make the invention thoroughly understood. The application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail as followed. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

The invention is to provide an alga essence nucleic acid fluid concentration method, to eliminate traditional direct heating method, but to extract the concentration by carry out the freezes, and defrost operation, this method may further achieve the simplification and maintain establishing of the production equipment, and then reduces the production cost, the concentrates extract of the chlorella essence nucleic acid fluid by this invention, may maintain its sweet flavor, and to improve the luster by join the tradition facture to obtained the concentrated chlorella essence.

The alga nucleic acid fluid referred in this invention is a kind of chlorella extracted from hot water concentration, the ingredient of this extracts are the protein, the polysaccharide, the Vitamin and the mineral substance etc the aqueous solution, long-term edible has the function of adjust one's physique, and to improve health. Moreover, in the chlorella nucleic acid fluid's special nutrition ingredient the alga essence accelerate growth factor (CGF) has many biological activity function, therefore the alga hot water extraction receives special attention, therefore, to judge the chlorella nucleic acid fluid quality is fit or unfit, usually rests on its nature quality (luster) and takes it CGF quantity (density).

To determinate its content of chlorella CGF and its legal expression system act according to Taiwan Commodity Inspection Bureau the CNS4202 and N5134 stipulation method. Naturally raised alga its CGF content majorities are situated between 1.3˜2.5; it will be different along with the different kind of plants and growthraise condition. The alga nucleic acid fluid's CGF content by the nature most density indicated the absorbance quality under light wave length 260 nm, for example OD200 or OD400. (OD: Optical Density).

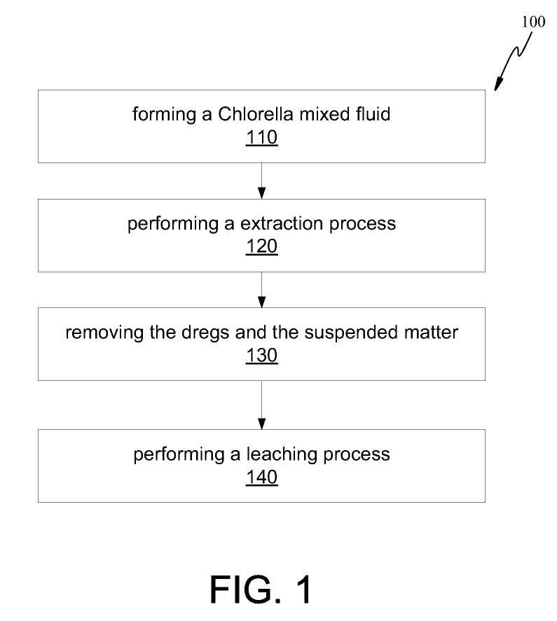

As shown in FIG. 1, a first embodiment of the present application discloses an alga essence nucleic acid fluid concentrated production method 100, comprising: forming a Chlorella mixed fluid 110, performing a extraction process 120, removing the dregs and the suspended matter and keeping the Chlorophyll in the Chlorella extract 130, and performing a leaching process 140. Depending on above description, forming a Chlorella mixed fluid 110, wherein the Chlorella mixed fluid is mixed and dampened by at least one of Chlorella raw materials and a mixed fluid. As for mixed liquid it can be water, secondary water, deionizer water or all nonpoisonous solvents etc. The above-mentioned the OD (Optical Density) value of the Chlorella raw materials is between 1.5 and 2.5. The Best, the OD (Optical Density) value of the Chlorella raw materials is approximately 2.3.

Continuously, an extraction process 120 is performed to heat the Chlorella mixed fluid evenly with first temperature and maintaining fixed time, and the heated Chlorella mixed fluid is to cooling down with the second temperature to form a Chlorella extract fluid. The above-mentioned first temperature is between 80 degree centigrade and 120 degree centigrade. As for being better, the above-mentioned first temperature is between 80 degrees centigrade to 95 degree centigrade. The above-mentioned chlorella mixed fluid is heated evenly at the first temperature, particular time is maintained, the particular time is between 30˜40 minutes. The above-mentioned second temperature is between 20 degree centigrade and 30 degree centigrade.

After the completing the extraction process 120, removing the dregs and the suspended matter and keeping the Chlorophyll in the Chlorella extract 130 to form an alga essence nucleic acid prototype product. Finally, a leaching process 140 is performed, the alga essence nucleic acid prototype product is frozen and then thawed naturally by leaching action to form a concentrated alga essence nucleic acid fluid. The above-mentioned concentrated alga essence nucleic acid fluid is more than or equal to 400. To repeat it at least 3 times as for relatively better ones with the above-mentioned leaching process 140.

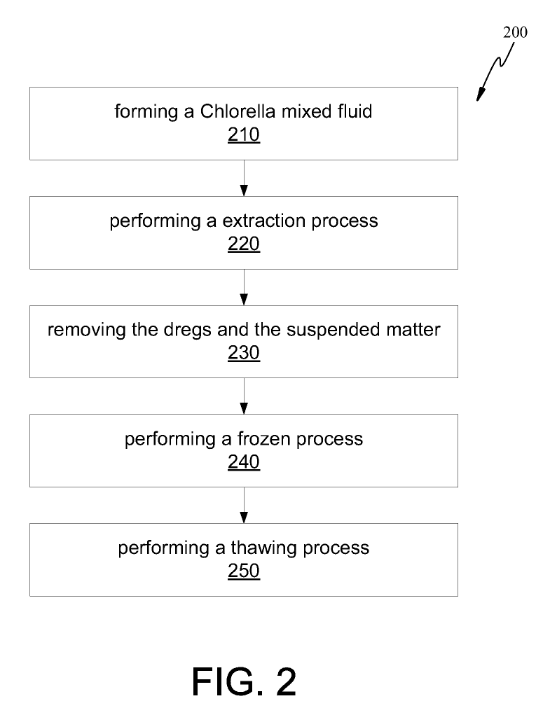

As shown in FIG. 2, a second embodiment of the present application discloses an alga essence nucleic acid fluid concentrated production method 200, comprising: forming a Chlorella mixed fluid 210, performing a extraction process 220, removing the dregs and the suspended matter and keeping the Chlorophyll in the Chlorella extract 230, performing a frozen process 240, and performing a thawing process 250. Depending on above description, forming a Chlorella mixed fluid 210, wherein the Chlorella mixed fluid is mixed and dampened by at least one of Chlorella raw materials and a mixed fluid. Then, an extraction process 220 is performed to heat the Chlorella mixed fluid evenly with first temperature and maintaining fixed time, and the heated Chlorella mixed fluid is to cooling down with the second temperature to form a Chlorella extract fluid. Next, removing the dregs and the suspended matter and keeping the Chlorophyll in the Chlorella extract 230 to form an alga essence nucleic acid prototype product. Next, a frozen process 240 is performed, the alga essence nucleic acid prototype product is frozen below freezing point of zero centigrade. Finally, a thawing process 250 is performed, the frozen alga essence nucleic acid fluid prototype product is naturally thawed approximately between 24 centigrade degree to 28 degrees to form said concentrated alga essence nucleic acid fluid. The above-mentioned chlorella raw materials OD value, first temperature, maintain a particular time at first temperature, second temperature, essence nucleic acid prototype product OD value and concentrated alga essence nucleic acid fluid OD value are same condition with the first embodiment of the present application.

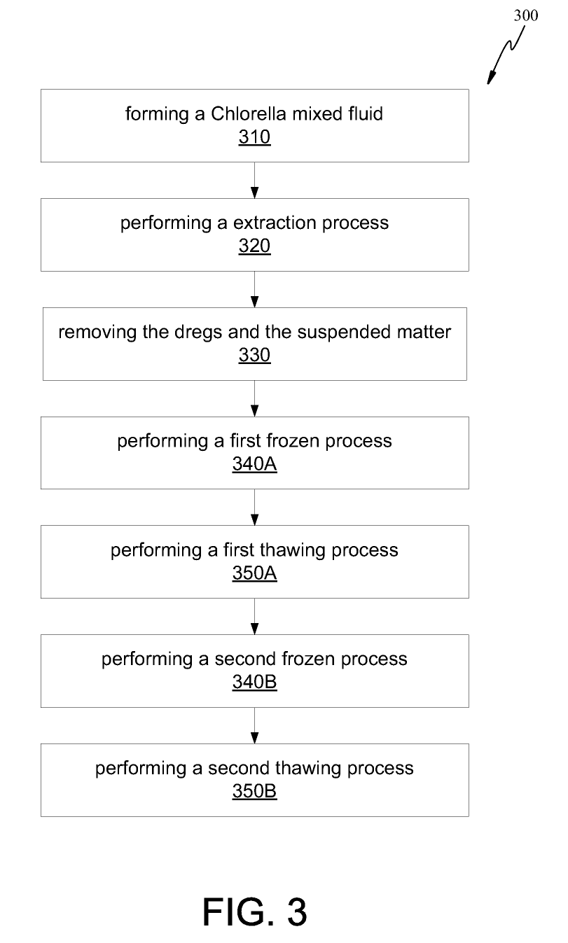

As shown in FIG. 3, a third embodiment of the present application discloses an alga essence nucleic acid fluid concentrated production method 300, comprising: forming a Chlorella mixed fluid 310, performing a extraction process 320, removing the dregs and the suspended matter and keeping the Chlorophyll in the Chlorella extract 330, performing a first frozen process 340A, performing a first thawing process 350A, performing a second frozen process 340B, and performing a second thawing process 350B. Depending on above description, forming a Chlorella mixed fluid 310, wherein the Chlorella mixed fluid is mixed and dampened by at least one of Chlorella raw materials and a mixed fluid. Then, an extraction process 320 is performed to heat the Chlorella mixed fluid evenly with first temperature and maintaining fixed time, and the heated Chlorella mixed fluid is to cooling down with the second temperature to form a Chlorella extract fluid. Next, removing the dregs and the suspended matter and keeping the Chlorophyll in the Chlorella extract 330 to form an alga essence nucleic acid prototype product. Next, a first frozen process 340A is performed, the alga essence nucleic acid prototype product is frozen below freezing point of zero centigrade. Next, a first thawing process 350A is performed, the frozen alga essence nucleic acid fluid prototype product is naturally thawed approximately between 24 centigrade degree to 28 degrees. Next, a second thawing process 340B is performed, the defrost alga essence nucleic acid fluid prototype product to freeze it again below the freezing point of the centigrade. Finally, a second thawing process 350B is performed, the frozen alga essence nucleic acid fluid prototype product is naturally thawed approximately between 24 centigrade degree to 28 degrees to form the concentrated alga essence nucleic acid fluid. The above-mentioned chlorella raw materials OD value, first temperature, maintain a particular time at first temperature, second temperature, essence nucleic acid prototype product OD value and concentrated alga essence nucleic acid fluid OD value are same condition with the first embodiment of the present application. The above-mentioned frozen process (340A; 340B) and with thawing process (350A; 350B) in between it can proceed to repeat several time.

### EXAMPLE 1

With regular chlorella powder raw material (Chlorella sp. ) and adds on 1:8˜10 time (w/w) water to infiltration mix with the alga raw material powder, to evenly add heating process until reach to 90˜100° C., and maintains 50˜60 minutes then carries on to cool downit to the normal temperature (room temperature), will obtain the mixture and after the separation of the solid material (wreckage of algae body), and after the steps of extracting suspension to keep the chlorophyll etc, it clarifies the amber leaning green liquid, namely the chlorella extracted liquid include the alga essence accelerate growth factor (CGF), I.e. the alga essence of nucleic acid fluid has not concentrated is the initial prototypeend product (the usual OD value situated between 130˜170), placeput this has not yet concentrated alga essence nucleic acid fluid of the prototype initial end product into a regular vessel, store it in the refrigerator to freeze it, this frozen prototypeinitial end product of un-concentrated alga essence nucleic acid fluid, take it out from the refrigerator and causes it to dissolve under the room temperature, because of the dissolved state continues to carry on (coexistent and mix with a solid condition) has the leaching function, After gathering this dissolution, the liquid state part (to be called leaching fluid or drop filtrate liquates), namely the concentrated alga essence of nucleic acid fluid; the un-dissolved portion content extremely few solute; may get rid of it or to redo it, to repeat the above step.

So-called “the leaching function” is refers to the ice in the initial dissolve liquid state portion, the solute content is much higher than average content in the ice solute; the ice dissolves the water is the function to cause the chemical composition to have the migration phenomenon in the ice.

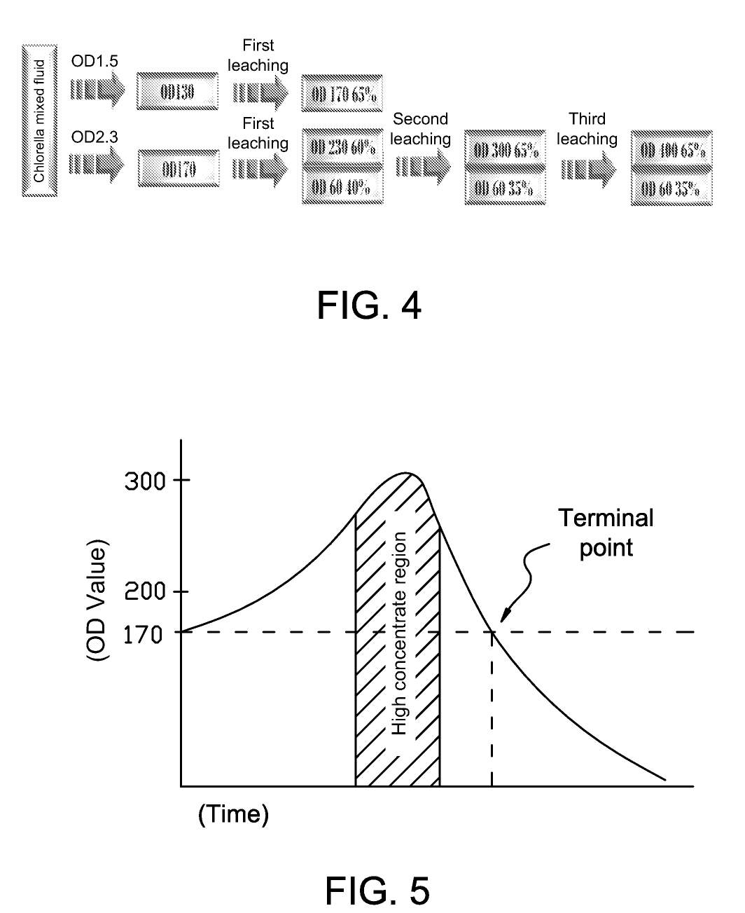

As show in FIG. 4, to operate the above steps repeatedly altogether by 3 times, at the prototype product of alga essence nucleic acid fluid it OD value is 170, it may be concentrates the OD value over above 400 of the green alga nucleic acid fluid.

In which, takes the raw material of chlorella to target for OD value 1.5, after depends on above step to manufacture, obtains the OD130 alga essence nucleic acid fluid at the prototype initial end product, after carry out the first freeze, defrosting, and the collection procedure, to obtains the density is above OD170; if this raw material chlorella OD value target is 2.3, after depend on above manufacture step, obtains the initial prototypeend product of the alga essence nucleic acid fluid at OD170, after carries on again to freeze it, defrosting, withand the collection procedure, the final product density of the OD value is above 230, repeatedly operates the above freezing, the dissolvinged and collection steps reaches to 3 times, the alga essence nucleic acid fluid may result in the OD value is above 400.

The concentration method apply to the production line operation has the extremely good flexibility, and ease to operate, the prototype initial end product of this alga essence nucleic acid fluid and place itat put in a regular 25˜35 liter barrel, place it in the refrigerator to freezecause it freeze, and take out this frozen prototype initial end product of alga essence nucleic acid fluid from the refrigerator and inverts it, causes it to dissolve naturally under the room temperature, continues to carry on with the dissolved state to forms the liquid-solid coexistent condition, to produce the leaching function, and then to collecting this leaching fluid (or calls drop filtrate), namely alga essence nucleic acid fluid from this invention.

As show in FIG. 5, when carries on the concentration step again with this invention, is also works as the leaching function that to produce, continually dissolves in the process, during various stages the collection of leaching fluid (or called drop filtrate) is the solute content and the time relational graph.

### EXAMPLE 2

The Food Analysis Center of Japan in Aug. 6, 2007 (Heisei 19) analyzed assay with result report based on the concentrated alga essence nucleic acid fluid report send in for exam arranged by the applicant, as show in below:

TABLE 1
Analytical Investigation Result

| | | Detection | | |
|---|---|---|---|---|
| Assay Items | Result | limit | Note | Method |
| Moisture | 97.4 g/100 g | | | Normal pressure |
| | | | | heating drying method |
| Protein | 1.6 g/100 g | | 1 | Kjeldahl method |
| Lipid | 0.1 g/100 g | | | Acid resolution |
| | Under | | | |
| Ash | 0.4 g/100 g | | | Directly ashing |
| | | | | method |
| Carbohydrate | 0.6 g/100 g | | 2 | |
| Energy | 9 kcal/100 g | | 3 | |

Note 1. Nitrogen, protein conversion factor: 6.25
Note 2. Formula: 100 − (Moisture + Protein + Lipid + Ash)
Note 3. Nutrition indicatory standard [Heisei 15(2003) Public Welfare Ministry of Labor Public Notice No. 176] About energy conversion factor: Protein, 4; Lipid, 9; Carbohydrate, 4

TABLE 2
Analytical Investigation Result

| | | Detection | | |
|---|---|---|---|---|
| Assay Items | Result | limit | Note | Method |
| Moisture | 97.4 g/100 g | | | Normal pressure |
| | | | | heating drying method |
| Protein | 1.6 g/100 g | | 1 | Kjeldahl method |
| Lipid | 0.1 g/100 g | | | Acid resolution |
| | Under | | | |
| Ash | 0.4 g/100 g | | | Directly ashing method |
| Sugar | 0.4 g/100 g | | 2 | |
| Dietary Fiber | 0.2 g/100 g | | | Enzyme - Gravimetric |
| | | | | method |
| Energy | 8 kcal/100 g | | 3 | |

Note 1. Nitrogen, protein conversion factor: 6.25
Note 2. Nutrition indicatory standard [Heisei 15(2003) Public Welfare Ministry of Labor Public Notice No. 176] About Formula: 100 − (Moisture + Protein + Lipid + Dietary Fiber)
Note 3. Nutrition indicatory standard [Heisei 15(2003) Public Welfare Ministry of Labor Public Notice No. 176] About energy conversion factor: Protein, 4; Lipid, 9; Suger, 4; Dietary fiber, 2

TABLE 3
Analytical Investigation Result

| Assay Items | Result | Detection limit | Note | Method |
|---|---|---|---|---|
| Sodium | 2.7 mg/100 g | | | Atom extinction brightness method ICP |
| Iron | 0.44 mg/100 g | | | luminous analysis |
| Calcium | 9.9 mg/100 g | | | ICP luminous analysis |
| Kalium | 117 mg/100 g | | | Atom extinction brightness method ICP |
| Magnesium | 20.5 mg/100 g | | | luminous analysis |
| Zinc | 0.14 mg/100 g | | | ICP luminous analysis |
| Selenium | Not detect | 5 μg/100 g | | Fluorescent brightness method |
| Entire chrome | Not detect | 0.05 mg/100 g | | ICP luminous analysis |
| Vitamin A(Retinol equivalence) | 3 μg/100 g | | 1 | |
| α-Carotin | 8 μg/100 g | | | High speed liquid chromatograph method |
| β-Carotin | 27 μg/100 g | | | High speed liquid chromatograph method |
| Thiamine (Vitamin B¹) | 0.03 mg/100 g | | 2 | High speed liquid chromatograph method |
| Ribo Flavin (Vitamin B²) | 0.42 mg/100 g | | | High speed liquid chromatograph method |
| VitaminB6 | 0.13 mg/100 g | | 3 | Bio assay method |
| VitaminB¹² | 1.1 μg/100 g | | 4 | Bio assay method |
| Ascorbic acid(Total Vitamin C) | Not detect | 1 mg/100 g | 5 | High speed liquid chromatograph method |
| Vitamin D | Not detect | 0.1 μg/100 g | | High speed liquid chromatograph method |
| Vitamin D (International unit) | — | | | |
| Vitamin E [symbol] | Not detect | | | High speed liquid chromatograph method |
| Folic acid | 82 μg/100 g | 0.1 mg/100 g | 6 | Bio assay method |

—: Because it does not detect, it does not calculate. Induction embody it measured
Note 1. α-Carotin 24 μg and β-Carotin 12 μg, respectively take the retinol equivalence of 1 μg
Note 2. As a thiamine salt
Note 3. Fungus strain used: Saccharomyces cerevisiae (S. uvarum) ATCC 9080
Note 4. Fungus strain used: Lactobacillus delbrueckii subsp. Lactis (L. leichmannii) ATCC 7830
Note 5. Apply hydrazine and measured after changes into induction embody.
Note 6. Fungus strain used: Lactobacillus rhamnosus (L. casei) ATCC 7469

TABLE 4
Analytical Investigation Result

| Assay Items | Result | Detection limit | Note | Method |
|---|---|---|---|---|
| Punting ten | 0.19 mg/100 g | | 1 | Bio assay method |
| Biotin | 5.6 μg/100 g | | 1 | Bio assay method |
| Inositol | 5 mg/100 g | | 2 | Bio assay method |
| Niacin | 3.02 mg/100 g | | 3 | |
| Niacin(Nicotine suitable quantity) | 2.85 mg/100 g | | 1 | Bio assay method |
| Toriputohuan | 10 mg/100 g | | | High speed liquid chromatograph method |
| Choline | Not detect | 0.03 g/100 g | 4 | |
| Dextro glucose | 0.22 g/100 g | | 5 | High speed liquid chromatograph method |
| Arabinose | Not detect | 0.02 g/100 g | 5 | High speed liquid chromatograph method |
| Xylose | Not detect | 0.02 g/100 g | 5 | High speed liquid chromatograph method |
| Rhamnose | 0.04 g/100 g | | 5 | High speed liquid chromatograph method |
| Mannose | Not detect | 0.02 g/100 g | 5 | High speed liquid chromatograph method |
| Galactose | 0.05 g/100 g | | 5 | High speed liquid chromatograph method |
| Lysine | 66 mg/100 g | | | Amino acid automatic analysis |
| Alanine | 112 mg/100 g | | | Amino acid automatic analysis |
| Glycine | 69 mg/100 g | | | Amino acid automatic analysis |
| Proline | 57 mg/100 g | | | Amino acid automatic analysis |
| Glutamic acid | 169 mg/100 g | | | Amino acid automatic analysis |
| Ceric | 45 mg/100 g | | | Amino acid automatic analysis |
| Aspartic acid | 99 mg/100 g | | | Amino acid automatic analysis |

Note 1. Fungus strain used: Lactobacillus plantarum ATCC 8014
Note 2. Fungus strain used: Saccharomyces cerevisiae (S. uvarum) ATCC 9080
Note 3. Niacin(Nicotine suitable quantity) and 1/60 of [symbol] total quantity was made by niacin equivalent
Note 4. Based on [symbol] salt settling method. The room temperature mixes the approximately 1 hour later
Note 5. Acid adding water to disassembled it and then measured. Hydrolysis condition: 72% of Sulfuric acid, after agitating it for one hour at room temperature, 4% sulfuric acid, put in autoclave (121° C.), one hour

TABLE 5
Analytical Investigation Result

| | | Detection | | |
|---|---|---|---|---|
| Assay Items | Result | limit | Note | Method |
| Total | 3.0 mg/100 g | | | Extinction brightness |
| Chlorophyll | | | | method (Visible) |
| Chlorophyll a | 2.7 mg/100 g | | | |
| Chlorophyll b | 0.3 mg/100 g | | | |
| Cobalt | Not detect | 0.05 ppm | | Atom extinction |
| | | | | brightness method |
| Germanium | Not detect | 1 ppm | | ICP luminous analysis |

Other modifications and variations are possibly developed in light of the above demonstrations. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. An alga essence nucleic acid fluid concentration method, comprising:
forming a Chlorella mixed fluid, wherein said Chlorella mixed fluid is formed by at least one of Chlorella raw materials and a mixed fluid, dampening it to succeed;
performing a extraction process, heating said Chlorella mixed fluid evenly with first temperature and maintaining fixed time, and said heated Chlorella mixed fluid is to cooling down with the second temperature to form a Chlorella extract fluid;
removing the dregs and the suspended matter and keeping the Chlorophyll in said Chlorella extract to form an alga essence nucleic acid prototype product; and
performing a leaching process, said alga essence nucleic acid prototype product is frozen and then thawed naturally by leaching action to form a concentrated alga essence nucleic acid fluid.

2. The alga essence nucleic acid fluid concentration method according to claim 1, wherein said first temperature is between 80 centigrade degrees to 120 degree.

3. The alga essence nucleic acid fluid concentration method according to claim 1, wherein said first temperature is between 85 centigrade degrees to 95 degree.

4. The alga essence nucleic acid fluid concentration method according to claim 1, wherein said Chlorella mixed fluid is heated to first temperature evenly, as though it to maintain certain time is between 30˜40 minute.

5. The alga essence nucleic acid fluid concentration method according to claim 1, wherein said second temperature is between 20 centigrade degrees to 30.

6. The alga essence nucleic acid fluid concentration method according to claim 1, wherein the OD (Optical Density) value of said Chlorella raw materials is between 1.5 and 2.5.

7. The alga essence nucleic acid fluid concentration method according to claim 1, wherein the OD (Optical Density) value of said Chlorella raw materials is approximately 2.3.

8. The alga essence nucleic acid fluid concentration method according to claim 1, wherein said the OD (Optical Density) value of said alga essence nucleic acid prototype product is more than or equal to 150.

9. The alga essence nucleic acid fluid concentration method according to claim 1, wherein said concentrated alga essence nucleic acid fluid is more than or equal to 400.

10. The alga essence nucleic acid fluid concentration method according to claim 1, wherein said leaching process further comprising:
performing a frozen process, said alga essence nucleic acid prototype product is frozen below freezing point of zero centigrade; and
performing a thawing process, the frozen alga essence nucleic acid fluid prototype product is naturally thawed approximately between 24 centigrade degree to 28 degrees to form said concentrated alga essence nucleic acid fluid.

11. The alga essence nucleic acid fluid concentration method according to claim 1, wherein said leaching process further comprising:
performing a first frozen process, said alga essence nucleic acid prototype product is frozen below freezing point of zero centigrade;
performing a first thawing process, the frozen alga essence nucleic acid fluid prototype product is naturally thawed approximately between 24 centigrade degree to 28 degrees;
performing a second frozen process, said alga essence nucleic acid fluid prototype product is frozen below freezing point of zero centigrade; and
performing a second thawing process, the frozen alga essence nucleic acid fluid prototype product is naturally thawed approximately between 24 centigrade degree to 28 degrees to form said concentrated alga essence nucleic acid fluid.

12. The alga essence nucleic acid fluid concentration method according to claim 1 to form an alga essence nucleic acid fluid, comprising:
a vegetable protein;
a lipid;
a Chlorophyll; and
several kinds of microelement.

13. The alga essence nucleic acid fluid concentration method according to claim 12, wherein said first temperature is between 80 centigrade degrees to 120 degree.

14. The alga essence nucleic acid fluid concentration method according to claim 12, wherein said first temperature is between 85 centigrade degrees to 95 degree.

15. The alga essence nucleic acid fluid concentration method according to claim 12, wherein said Chlorella mixed fluid is heated to first temperature evenly, as though it to maintain certain time is between 30˜40 minute.

16. The alga essence nucleic acid fluid concentration method according to claim 12, wherein the OD (Optical Density) value of said Chlorella raw materials is between 1.5 and 2.5.

17. The alga essence nucleic acid fluid concentration method according to claim 12, wherein the OD (Optical Density) value of said Chlorella raw materials is approximately 2.3.

18. The alga essence nucleic acid fluid concentration method according to claim 12, wherein said the OD (Optical Density) value of said alga essence nucleic acid prototype product is more than or equal to 150.

19. The alga essence nucleic acid fluid concentration method according to claim 12, wherein said concentrated alga essence nucleic acid fluid is more than or equal to 400.
